(19) United States
(12) Patent Application Publication
(10) Pub. No.: US 2005/0031690 A1
(43) Pub. Date: Feb. 10, 2005

(54) Stabilized prostaglandin formulation

(75) Inventors: Brian R. Rohrs, Scotts, MI (US); Scott L. Douglas, Richland, MI (US); John M. Heimlich, Portage, MI (US); James F. Miller, Morpeth Northumbreland (GB); George A. Burgess, Dinnington Newcastle-upon Tyme (GB); Philip J. Rolfe, Morpeth Northumbreland (GB)

Correspondence Address: PHARMACIA CORPORATION;GLOBAL PATENT DEPARTMENT, POST OFFICE BOX 1027, ST. LOUIS, MO, 63006, US

(73) Assignee: Pharmacia Corporation

(21) Appl. No.: 10/825,397

(22) Filed: Apr. 15, 2004

Related U.S. Application Data
(60) Provisional application No. 60/463,356, filed on Apr. 16, 2003.

(51) Int. Cl. A61K 31/60; A61K 31/557; A61K 9/24; A61K 9/28

(52) U.S. Cl. 424/474; 514/165; 514/573

(57) ABSTRACT

A discrete solid orally deliverable pharmaceutical dosage form comprises a plurality of zones, wherein (a) at least one zone comprises an NSAID; (b) at least one zone, other than a zone comprising the NSAID, comprises HPMC having dispersed therein a prostaglandin type compound in a form of a substantially water-free solid dispersion; (c) the plurality of zones are spatially arranged such that, if there is only one NSAID-containing zone and one prostaglandin-containing zone, these zones are arranged other than as a core and mantle respectively separated by an enteric coating layer; and (d) the HPMC comprises a fraction having particle size smaller than about 53 μm, said fraction exhibiting, upon dissolution in C0₂-free purified water to form a 1% weight/volume solution, a pH not lower than about 4. An assay method is also provided for selecting suitable lots of HPMC for use in preparing such a dosage form.

This application claims priority of U.S. provisional application Ser. No. 60/463,356 filed Apr. 16, 2003.

### FIELD OF THE INVENTION

This invention relates to formulations of prostaglandin drugs, for example misoprostol, and in particular to such formulations wherein the drug is dispersed in a polymer matrix. This invention has especial relevance to coformulations of a prostaglandin and a nonsteroidal anti-inflammatory drug (NSAID).

### BACKGROUND OF THE INVENTION

Prostaglandin type compounds, particularly prostaglandin E₁ derivatives such as misoprostol (I), which are typically viscous, oily liquids have long been formulated as solid dispersions in a polymer matrix.

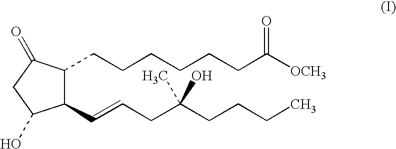

For example, U.S. Pat. No. 4,301,146 to Sanvordeker discloses a solid dispersion comprising 1 part of misoprostol to about 50 to about 500 parts of a polymer. The polymer used is either hydroxypropylmethylcellulose (HPMC) or polyvinylpyrrolidone (PVP). The solid dispersion is said to be suitable for filling into capsules or compressing into tablets in a conventional manner. Improved chemical stability of the misoprostol in an HPMC dispersion by comparison with misoprostol alone is reported, particularly at elevated temperature.

U.S. Pat. No. 5,889,051 to Chen et al. discloses a solid dispersion of misoprostol in an ammonio methacrylate copolymer, that is said to provide sustained release of the misoprostol.

U.S. Pat. No. 5,935,939 to Kararli et al. discloses a solid dispersion of misoprostol in one or more amorphous excipients or excipients that have been converted to an amorphous state.

Chemical degradation of certain prostaglandin type compounds, particularly prostaglandin E₁ derivatives such as misoprostol, is accelerated in presence of water, and the primary pathway of degradation is believed to be dehydration to the corresponding prostaglandin A derivative. The problem of chemical instability becomes more acute when the prostaglandin type compound is coformulated with certain NSAIDs such as diclofenac or piroxicam.

Coformulations of an NSAID with a prostaglandin type compound, for example prostaglandin E₁ or a derivative thereof such as misoprostol, are highly desired in the art. NSAIDs present great therapeutic benefit in treatment of inflammatory conditions such as arthritis, but have an ulcerogenic effect in the upper gastrointestinal tract which can seriously limit their usefulness, especially for chronic treatment. Certain prostaglandin type compounds, especially prostaglandin E₁ derivatives and more particularly misoprostol, have been found to mitigate or provide protection against such ulcerogenic effects when co-administered with an NSAID. Arthrotec® tablets of Pharmacia Corporation, comprising diclofenac sodium (50 or 75 mg) and misoprostol (0.2 mg) are an example of a coformulated drug product combining the powerful anti-inflammatory effect of an NSAID with the gastroprotective effect of a prostaglandin. See Physicians' Desk Reference, 57th edition (2003), 3103-3107.

U.S. Pat. No. 5,015,481 to Franz et al. discloses a pharmaceutical composition comprising an admixture of an NSAID selected from diclofenac and piroxicam, a prostaglandin such as misoprostol, and a stabilizer, preferably HPMC. It is reported therein that HPMC provides an especially useful stabilizing effect on the prostaglandin component in the presence of the NSAID component.

U.S. Pat. No.5,601,843 to Gimet et al. discloses a tablet having a core surrounded by a mantle. The core comprises an NSAID and the mantle comprises a prostaglandin such as misoprostol, for example in a form of a solid dispersion in a polymer such as HPMC. An enteric coating is optionally present between the core and the mantle. The Arthrotec® product mentioned above is a tablet of this kind. By maintaining spatial separation of the NSAID and the prostaglandin as in a core-and-mantle tablet, it is believed that further improvement in chemical stability of the prostaglandin is achievable.

European Patent Application No. 1 068 867 of Sherman also discloses a tablet having an NSAID in the core and a prostaglandin, dispersed in a polymer such as HPMC, in a zone surrounding the core. In this case the surrounding zone is referred to as a film coating rather than a mantle.

Other dosage form configurations maintaining spatial separation of NSAID and prostaglandin are possible. For example, European Patent Application No. 1 020 182 of Sherman discloses a two-layer tablet having an NSAID and misoprostol located in separate layers. Again the misoprostol can be in a form of a solid dispersion in HPMC.

International Patent Publication No. WO 99/65496 of Sherman discloses a pharmaceutical tablet that incorporates two smaller tablets, one of which comprises an NSAID and the other of which comprises misoprostol, preferably in a form of a solid dispersion in HPMC.

International Patent Publication No. WO 00/01368 of Norton Healthcare and U.S. Pat. No. 6,319,519 to Woolfe et al. each discloses a dosage form wherein an NSAID is located in coated pellets and misoprostol, for example in a form of a solid dispersion in HPMC or PVP, is located outside the pellets. A similar dosage form is proposed in U.S. Pat. No. 6,183,779 and in U.S. Pat. No. 6,287,600, both to Ouali & Azad, except that the NSAID is included in the form of enterically coated granules or particles. In International Patent Publication No. WO 00/15200 of Norton Healthcare and U.S. Pat. No. 6,387,410 to Woolfe et al., a similar dosage form is disclosed except that the NSAID containing pellets are said to be in a delayed release formulation.

Most of the above-cited publications disclose a 1:100 dispersion of misoprostol in HPMC. Although HPMC is known to exist in a wide variety of grades varying in molecular weight, viscosity and degree of methoxy and hydroxypropoxy substitution (see Handbook of Pharmaceutical Excipients, 3rd edition (2000), 252-255), no guidance is provided in any of the above-cited publications as to preferred HPMC properties for stabilization of misoprostol or other prostaglandins.

Stability of some prostaglandins is known to be pH-sensitive. In a solution state, prostaglandin E₁ has been reported to exhibit a region of relative stability at about pH 3-4. Monkhouse et al. (1973), J. Pharm. Sci. 62(4), 576-580. A similar pH effect on stability of misoprostol in a solution state has been reported by Toledo-Velasquez et al. (1992), J. Pharm. Sci. 81(2), 145-148. U.S. Pat. No. 4,335,097 to David et al. discloses a composition comprising a prostaglandin, specifically prostaglandin F_{2α}, and a buffer which adjusts pH of a liquid film, formed on the surface of the solid phase due to air humidity, to 3 to 5.

In an HPMC matrix, prostaglandin degradation is, as indicated above, substantially reduced but is not necessarily eliminated. There is a need for long shelf life, for example up to 2 years, for drug products. Drug products can readily be protected from high relative humidity conditions, for example by moisture barrier packaging, but protection from high temperature exposure is less readily assured. Like most chemical degradation processes, dehydration of prostaglandin type compounds such as misoprostol is accelerated at elevated temperatures. Thus even where degradation is substantially reduced by formulation of the prostaglandin type compound as a solid dispersion in HPMC, there remains a need for further improvement in long-term chemical stability, especially in coformulations of the prostaglandin/HPMC dispersion with an NSAID.

It is believed that this need is particularly great where such coformulation is presented in a form other than a core-and-mantle tablet with an enteric coating layer between the core and mantle, such as the Arthrotec® product mentioned above.

### SUMMARY OF THE INVENTION

There is now provided a discrete solid orally deliverable pharmaceutical dosage form comprising a plurality of zones. At least one zone of the dosage form comprises an NSAID, the NSAID being present in a therapeutically effective total amount in the dosage form. At least one zone of the dosage form, other than a zone comprising the NSAID, comprises HPMC having dispersed therein a prostaglandin type compound in a form of a substantially water-free solid dispersion, the prostaglandin type compound being present in the dosage form in a total amount effective to mitigate a gastric ulcerogenic effect of the NSAID. The dosage form has a spatial arrangement of zones such that, if there is only one NSAID-containing zone and one prostaglandin-containing zone, such zones are arranged other than as a core and mantle respectively with an enteric coating layer therebetween. Importantly, the HPMC is selected or treated such that when fractionated by particle size, a fraction having particle size smaller than about 53 μm (herein the “sub-53 μm fraction”), upon dissolution in CO₂-free purified water to form a 1% weight/volume solution, exhibits a pH not lower than about 4.

A dosage form of the invention exhibits improved chemical stability of the prostaglandin component by comparison with an otherwise similar dosage form wherein the HPMC, upon fractionation and dissolution of the sub-53 μm fraction as described above, exhibits a pH lower than 4. This finding is surprising at least for the reason that prostaglandin stability in a substantially water-free HPMC dispersion has not previously been known to be affected by pH (as measured upon dissolution in water) of any fraction of the HPMC. Indeed, it is not known by what mechanism such pH can influence prostaglandin stability in the substantial absence of water.

Many commercially available HPMCs have been found to exhibit a pH lower than 4 when tested in accordance with the fractionation and dissolution test provided herein. Such HPMCs are, surprisingly, unsuitable for use in a prostaglandin/NSAID formulation as contemplated in the present invention. By identifying a class of HPMCs enabling greater prostaglandin stability, and by providing a test for determining whether a given lot of HPMC falls within that class, an important advance in the art has been made.

### DETAILED DESCRIPTION OF THE INVENTION

The pharmaceutical dosage form of the present invention is a discrete solid orally deliverable dosage form such as a tablet, caplet, pill, pellet, hard or soft capsule, lozenge or troche. The term “orally deliverable” herein means suitable for administration via the mouth, e.g., peroral, buccal or sublingual administration, but preferably the dosage form is adapted for delivery per os, in other words by placement in the mouth followed by swallowing of the discrete solid dosage form, typically with the aid of water or other liquid. More preferably the dosage form is suitable for swallowing whole.

The dosage form comprises a plurality of zones, at least one of which comprises an NSAID and another of which comprises a solid dispersion of a prostaglandin type compound in HPMC. In one embodiment the dosage form comprises a single NSAID-containing zone and a single prostaglandin-containing zone, these zones being disposed other than as an NSAID-containing core surrounded by a prostaglandin-containing mantle with an enteric coating layer between the core and the mantle. Illustratively, the two zones can be disposed as follows:

(a) as two layers of a bilayer tablet;

(b) as two compartments of a dual-compartment capsule;

(c) as two pre-compressed or pre-molded tablets embedded within a single larger dosage form;

(d) as a prostaglandin-containing core surrounded by an NSAID-containing mantle; or

(e) as an NSAID-containing core surrounded by a prostaglandin-containing mantle, wherein the core and mantle are not separated by an enteric coating layer.

In another embodiment the dosage form comprises more than two zones, wherein at least one of the NSAID and the prostaglandin components is present in more than one zone. Illustratively, these zones can be disposed as follows:

(f) as layers of a multilayer tablet, for example having two outer prostaglandin-containing layers having between them a middle NSAID-containing layer in a sandwich arrangement;

(g) as a plurality of separately NSAID-containing and prostaglandin-containing particles compressed or molded into a single tablet, wherein the term “particles” embraces granules, beads, individual particles in a multiparticulate formulation, etc.;

(h) as compartments of a multi-compartment capsule;

(i) as a plurality of separately NSAID-containing and prostaglandin-containing beads in a capsule; or

(j) as a plurality of beads, at least a fraction of which individually comprise a core comprising the NSAID surrounded by a mantle comprising the prostaglandin type compound.

Regardless of the precise disposition of the NSAID-containing and prostaglandin-containing zones relative to one another, it is preferred that a barrier layer be present between the zones, having the effect of substantially preventing contact of the NSAID with the prostaglandin type compound. Such a barrier layer can be, for example, a coating on the NSAID-containing and/or prostaglandin-containing zones; in a preferred embodiment the NSAID-containing zone or zones are provided with an enteric coating. Alternatively, in a bilayer tablet for example, the barrier layer is present only between the NSAID-containing layer and the prostaglandin-containing layer. As yet another alternative, the barrier layer takes the form of a matrix wherein NSAID-containing and prostaglandin-containing granules or beads are dispersed. The barrier layer prevents or minimizes risk of contact between the NSAID and the prostaglandin type compound prior to oral administration of the dosage form, and preferably during and after such oral administration.

In the prostaglandin-containing zone or zones, the prostaglandin type compound is dispersed in a substantially water-free solid dispersion in a matrix comprising HPMC having a property described herein as “low residual acidity”. An HPMC lot having this property is described herein as a “low residual acid HPMC”. The HPMC can have low residual acidity as supplied by the manufacturer, or it can be treated to become a low residual acid HPMC. Such treatment can occur prior to preparing the dosage form or it can be a part of the process of preparing the dosage form, as more fully explained hereinbelow.

The following test (Test I) can be used to determine whether a given lot of HPMC has low residual acidity as required by the present invention. It will be noted that “low residual acidity” as defined herein is not determinable simply by measuring pH of a bulk sample of HPMC dissolved in water.

Test I

1. A sample of the HPMC to be tested is fractionated by particle size, for example in a sieving operation, to provide a fraction having particle size smaller than about 53 μm (the “sub-53 μm fraction”). For example, particles that pass through a U.S. standard 270 mesh screen can be considered the sub-53 μm fraction for the purposes of this test.

2. CO₂-free purified water is prepared as a dissolution medium for pH determination. Purified water meeting pharmacopeial standards, e.g., U.S.P., is suitable when freshly boiled to remove any dissolved carbon dioxide. A pH measurement of the CO₂-free purified water taken immediately before use should be in the range of 6.0-7.0.

3. A suitable amount of CO₂-free purified water is placed in a beaker or other suitable container and heated to about 90° C. For example, 50 ml of CO₂-free purified water in a 150 ml beaker will be found suitable.

4. A precisely weighed amount of the sub-53 μm fraction of the HPMC sample is transferred to the CO₂-free purified water in the beaker with continuous stirring. An amount of exactly 1.0 g HPMC will be found suitable.

5. The beaker and its contents are allowed to cool to room temperature (20-25° C.), and the contents are diluted with CO₂-free purified water to provide a 1% HPMC solution. For example, if 1.0 g HPMC has been used for the test, the contents of the beaker are diluted to 100 ml. Stirring is continued until all solid material has dissolved.

6. The pH of the resulting solution is measured using a previously calibrated pH meter. The electrode of the pH meter must be immersed in the solution and the pH reading must be stable before being recorded. Covering the sample beaker with aluminum foil may assist in obtaining a steady pH reading.

7. A pH of 4.0 or higher indicates an HPMC sample of low residual acidity as required by the present invention.

Preferably, the HPMC used in a dosage form of the invention exhibits a pH not lower than about 4.5, more preferably not lower than about 5, and most preferably not lower than about 6, in the above test.

The present invention is not limited by the process used to prepare the HPMC or the method used to achieve low residual acidity. However, it is believed that one way in which the problem of low pH (as measured in the above test) can arise is related to the manufacturing process for HPMC. In at least one such process, cellulose polymer is subjected to acid hydrolysis, for example using gaseous hydrogen chloride, to reduce polymer chain length and thereby control molecular weight and viscosity of the resulting HPMC. Excess acid is neutralized by adding a pH modifying agent, typically a base, for example in the form of solid particles of sodium carbonate or sodium bicarbonate. If an insufficient amount of pH modifying agent is added, the resulting HPMC can exhibit a pH lower than 4 in the above test and will then be unsuitable, without further treatment, for use in a dosage form of the invention.

Processes wherein excess acid, resulting from acid hydrolysis of HPMC to reduce polymer chain length, is neutralized by adding a base are illustratively disclosed in U.S. Pat. No. 3,391,135 to Ouno et al. and U.S. Pat. No. 4,061,859 to Cheng, both of which are incorporated herein by reference.

Even if a sufficient amount of a solid particulate base has been used to neutralize excess acid, the resulting HPMC can still fail the above test. It is believed, without being bound by theory, that if particle size of the base is too large, pH of the sub-53 μm fraction of the HPMC can remain low even where a theoretically adequate amount of base has been added. In a situation such as this, milling of the HPMC to reduce particle size of the base therein can be helpful in achieving a low residual acid HPMC as required by the present invention.

Addition of a pH modifying agent, for example a base, preferably a solid base such as sodium carbonate or sodium bicarbonate in an amount of about 0.01% to about 5% of the HPMC, as part of the process of preparing a dosage form of the invention, can be effective in converting an unsuitable HPMC lot into one having the required low residual acidity. Preferably such addition is made prior to dispersing the prostaglandin in the HPMC.

Vacuum drying of an HPMC sample before use can also be beneficial in this regard. It is believed that vacuum drying drives off volatile residual hydrogen chloride present in the HPMC.

Other treatment methods to correct a problem of low residual acidity will be evident to one of skill in the art.

However, in a manufacturing setting it will generally be found more practical to pre-select HPMC lots having low residual acidity, by use of a test such as that provided above. The test can be implemented by the supplier of the HPMC and made part of the raw material specifications.

HPMC is available in a variety of types, differing for example in relative degree of methoxy and hydroxypropoxy substitution and in molecular weight. Any suitable HPMC type can be used so long as the particular lot has low residual acidity as defined herein. A presently preferred HPMC conforms to substitution type 2910 as defined in the United States Pharmacopeia, 24th Edition (2000), page 843.

The solid dispersion of the prostaglandin in the HPMC matrix is described herein as “substantially water-free”, which means that the moisture content of the dispersion is no greater than the equilibrium moisture content of HPMC at 80% relative humidity. Preferably the moisture content of the dispersion is no greater than about 11%, more preferably no greater than about 7%.

The prostaglandin type compound that is dispersed in the HPMC can be any pharmacologically active prostaglandin, prostacyclin or thromboxane or derivative thereof, or a prodrug thereof. Examples include without limitation alfaprostol, beraprost, carboprost, cloprostenol, enprostil, fenprostalene, fluprostenol, gemeprost, latanoprost, limaprost, luprostiol, misoprostol, ornoprostil, prostacyclin, prostaglandin E₁, prostaglandin E₂, prostaglandin F_{2α}, prostalene, rioprostil, rosaprostol, sulprostone, taprostene, thromboxane A₂, thromboxane B₂, tiaprost, trimoprostil, unoprostone, and salts, esters, tautomers, enantiomers and polymorphs thereof. Preferred are prostaglandins E₁ and E₂ and derivatives thereof, including enprostil, gemeprost, limaprost, misoprostol, ornoprostil, rioprostil and sulprostone. An especially preferred prostaglandin is misoprostol (I).

The invention is described herein with particular reference to misoprostol, but it will be understood that another prostaglandin type compound can be substituted for the misoprostol if desired.

Misoprostol is dispersed in the HPMC in a weight ratio of about 1:1000 to about 1:10, preferably about 1:500 to about 1:20, for example about 1:200 to about 1:50. Particularly suitable is a weight ratio of misoprostol to HPMC similar to that found in Arthrotec® tablets, namely about 1:99.

Misoprostol is present in the dosage form as a whole in an amount sufficient to mitigate a gastric ulcerogenic effect of the NSAID component. Typically such an amount is about 50 to about 400 μg, preferably about 100 to about 300 μg, per dosage form. A particularly suitable amount is similar to that found in Arthrotec® tablets, namely about 200 μg. If the amount is too low, the gastroprotective effect of the misoprostol can be inadequate; if the amount is too high, side effects such as diarrhea can result. Where another prostaglandin type compound is substituted for misoprostol, an amount therapeutically equivalent to that given herein for misoprostol should be used.

Other excipients can optionally be added to the misoprostol/HPMC dispersion, including conventional diluents, binders, dispersants, wetting agents, disintegrants, lubricants, preservatives, coloring and flavoring agents, etc.

Where misoprostol is formulated with an HPMC not meeting the criteria set forth herein, dehydration of the misoprostol to the corresponding prostaglandin A derivative, herein named the “A-form” of misoprostol (II), can occur to an unacceptable degree.

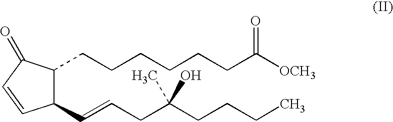

The following empirical test (Test II) can be used in a manufacturing plant as an alternative or supplement to Test I above to determine whether a particular lot of HPMC is suitable for use in preparing a dosage form of the invention.

Test II

1. A “control” lot of HPMC, known to produce a good quality product exhibiting a high degree of misoprostol stability, is identified, for example from quality assurance (QA) records. The average A-form content is computed from QA records of batches of product using the “control” lot of HPMC.

2. A solution of 12.5 mg/ml misoprostol in ethanol is prepared, for example by dissolving 0.625 g misoprostol in 50 ml ethanol. The solution is allowed to stand for about 30 minutes, with occasional shaking to ensure complete dissolution.

3. For each of a test sample of HPMC and a sample of the “control” HPMC, a 2 g amount is weighed and placed in a 100 ml conical flask fitted with a glass stirring rod, a PTFE paddle (inverted) and a stirrer gland. This step should be completed as quickly as possible to minimize risk of moisture ingress to the flask. The flask is transferred to a mechanical stirrer and the apparatus is checked to ensure smooth stirring and consequently good mixing.

4. The stirrer gland is raised to allow insertion of a pipette, from which is added 1.6 ml of the misoprostol solution, with continuous stirring. As soon as the misoprostol solution has been added, the pipette is withdrawn and the stirrer gland lowered to seal the flask. The resulting mixture is stirred for about 15 minutes to provide a damp cake.

5. The apparatus is then dismantled, but before the stirring rod and paddle are removed, as much as possible of the damp cake is scraped off. Damp cake is also scraped off the sides of the flask, breaking any large lumps that may have formed.

6. The flask is then stoppered and placed in an oven at 50° C. for about 16 hours.

7. Chromatographic analysis, e.g., high performance liquid chromatography (HPLC), for A-form misoprostol is performed on the whole sample.

8. The ratio of A-form content for the test sample to that for the “control” sample is calculated and this ratio is then multiplied by the average A-form content of the “control” HPMC as obtained in Step 1 above, to give a predicted A-form content for the HPMC lot being tested.

9. If the predicted A-form content is not greater than a specified maximum content (e.g., 0.21% A-form), the HPMC lot is deemed suitable for use.

In a variant of the above Test II, 1 g of HPMC is weighed into a 20 ml screw-cap glass vial. While stirring magnetically, 0.8 ml of a 12.5 mg/ml solution of misoprostol in absolute ethanol is added and the vial tightly capped. Magnetic stirring is continued for about 5 minutes. The vial is then briefly opened and damp cake is removed from the walls of the vial using a spatula. The vial is recapped and sealed tightly with Parafilm. The vial is then placed in an oven at 50° C. for 16 hours. HPLC analysis is carried out on the whole sample to eliminate potential heterogeneity problems. After cooling, 10 ml of acetonitrile is added to the sample and the resulting mixture is stirred for about 1 hour. Insoluble material is allowed to settle and the supernatant is then filtered through a 0.45 μm filter. A measured amount, for example 2 ml, of the filtrate is collected and evaporated to dryness using a stream of nitrogen. The dried filtrate is reconstituted in 1 ml of a suitable HPLC mobile phase, for example as described in Test III below, and assayed by HPLC. Concentration of misoprostol and of its A-form dehydration product is determined by comparing the peak area in each case to a series of reference standards.

Stability of misoprostol in a dosage form as described herein can be assessed by the following illustrative test (Test III).

Test III

1. One or more misoprostol-containing zone or zones, or parts thereof, are mechanically separated from the NSAID-containing zones of a dosage form.

For example, in the case of a bilayer tablet this can be done by cutting with a knife or splitting with pliers. Where a barrier layer such as an enteric coating is present between misoprostol-containing and NSAID-containing zones, the dosage form typically exhibits a tendency to break along the barrier layer, thus facilitating mechanical separation. A sample consisting of the misoprostol-containing zones of about 5 to about 10 dosage forms will typically suffice for the analysis that follows.

2. The sample is placed in a vial and a suitable volume, for example about 10 ml, of acetonitrile is added.

3. The contents of the vial are vortexed for 1-3 minutes and stirred for about 1 hour, to dissolve misoprostol and A-form dehydration product.

4. After standing for about 2 minutes to permit settling of HPMC and other excipient materials, the supernatant is withdrawn and filtered through a 0.45 μm filter.

5. A measured volume, for example 4 ml, of the filtrate is collected and evaporated to dryness using a stream of nitrogen.

6. The dried filtrate is reconstituted in 0.3 ml of a suitable HPLC mobile phase, for example as indicated below, and filtered again through a 0.22 μm centrifuge filter to remove trace amounts of insoluble material.

7. The resulting filtrate is assayed by HPLC. Suitable conditions are illustratively as follows:

| | |
|---|---|
| column | Luna, 5 μm, C8, 150 × 4.6 mm (Phenomenex) |
| mobile phase | 10/40/50 isopropanol/acetonitrile/water |
| flow rate | 1 ml |
| injection volume | 25 μl |
| detection | 200 nm |

Concentration of misoprostol and of its A-form dehydration product is determined by comparing the peak area in each case to a series of reference standards.

If the predicted A-form content is not greater than a specified maximum content (e.g., 0.21% A-form), the HPMC lot is deemed suitable for use.

In the NSAID-containing zone or zones of the dosage form, any NSAID can be used, including without limitation aceclofenac, acemetacin, ε-acetamidocaproic acid, acetaminosalol, S-adenosylmethionine, alclofenac, alminoprofen, amfenac, 3-amino-4-hydroxybutyric acid, ampiroxicam, amtolmetin guacil, apazone, aspirin, balsalazide, bendazac, benorylate, benoxaprofen, benzpiperylon, benzydamine, bermoprofen, α-bisabolol, bromfenac, bucolome, bufexamac, bumadizon, butibufen, carprofen, cinmetacin, clidanac, clopirac, diclofenac, difenamizole, difenpiramide, diflunisal, ditazol, droxicam, emorfazone, enfenamic acid, epirizole, etanercept, etodolac, etofenamate, felbinac, fenbufen, fenclozic acid, fendosal, fenoprofen, fentiazac, fepradinol, feprazone, flufenamic acid, flunoxaprofen, flurbiprofen, gentisic acid, glucametacin, glycol salicylate, guaiazulene, ibufenac, ibuprofen, ibuproxam, indomethacin, indoprofen, infliximab, isonixin, isoxepac, isoxicam, ketoprofen, ketorolac, lexipafant, lonazolac, lornoxicam, loxoprofen, meclofenamic acid, mefenamic acid, meloxicam, mesalamine, metiazinic acid, mofebutazone, mofezolac, morazone, nabumetone, 1-naphthyl salicylate, naproxen, niflumic acid, nimesulide, olsalazine, oxaceprol, oxametacine, oxaprozin, oxyphenbutazone, paranyline, parsalmide, perisoxal, phenyl acetylsalicylate, phenylbutazone, phenyl salicylate, piketoprofen, pipebuzone, pirazolac, piroxicam, pirprofen, pranoprofen, proglumetacin, propyphenazone, proquazone, protizinic acid, ramifenazone, salacetamide, salicylamide o-acetic acid, salicylic acid, salicylsulfuric acid, salsalate, sulfasalazine, sulindac, suprofen, suxibuzone, talniflumate, tenidap, tenoxicam, terofenamate, thiazolinobutazone, tiaprofenic acid, tiaramide, tinoridine, tolfenamic acid, tolmetin, tropesin, xenbucin, ximoprofen, zaltoprofen, zomepirac, and salts, esters, tautomers, enantiomers and polymorphs thereof.

Preferred NSAIDs include diclofenac and its pharmaceutically acceptable salts, for example diclofenac sodium, and piroxicam.

The NSAID is present in the dosage form as a whole in a therapeutically effective amount, more particularly an anti-inflammatorily effective amount. In the case of diclofenac or a salt thereof, typically such an amount is about 20 to about 200 mg, preferably about 40 to about 100 mg, per dosage form. A particularly suitable amount is similar to that found in Arthrotec® tablets, namely about 50 mg or about 75 mg. Where another NSAID is substituted for diclofenac, an amount therapeutically equivalent to that given herein for diclofenac should be used.

The NSAID can be formulated together with any suitable excipients, including conventional diluents, binders, dispersants, wetting agents, disintegrants, lubricants, preservatives, coloring and flavoring agents, etc.

### EXAMPLES

The following examples illustrate aspects of the present invention but are not to be construed as limitations.

### Example 1

Three lots of HPMC were tested according to the procedure of Test I above. The pH of unsieved HPMC was also determined for each lot. Data are shown inl Table 1.

TABLE 1

| Lot | pH (unsieved) | pH (sub-53 μm) | weight % (sub-53 μm) |
|---|---|---|---|
| A | 7.2 | 7.3 | 45 |
| B | 6.6 | 7.6 | 37 |
| C | 7.2 | 3.1 | 32 |

A dispersion of 1 part misoprostol in 99 parts HPMC was prepared using HPMC of each of Lots A, B and C. Lots A and B, having low residual acidity as defined herein, provided a dispersion exhibiting good misoprostol stability as measured by low A-form content following storage at 55° C. for 26 weeks. Lot C, having a high degree of residual acidity as indicated by a pH of the sub-53 μm fraction that was lower than 4, provided a dispersion exhibiting very poor misoprostol stability as measured by an unacceptably high A-form content following storage under the same conditions.

The poor performance of HPMC Lot C was not related to its bulk pH, i.e., the pH of unsieved HPMC.

### Example 2

A single lot of HPMC, known to result in poor misoprostol stability, was tested according to the procedure of Test I above, with and without pre-milling. Data are shown in Table 2.

TABLE 2

| Lot | pH (unsieved) | pH (sub-53 μm) | weight % (sub-53 μm) |
|---|---|---|---|
| D (unmilled) | 7.1 | 3.3 | 40 |
| D (milled) | 7.7 | 7.2 | 46 |

A dispersion of 1 part misoprostol in 99 parts HPMC was prepared using HPMC of each of the milled and unmilled samples. The unmilled HPMC, having a high degree of residual acidity as indicated by a pH of the sub-53 μm fraction that was lower than 4, provided a dispersion exhibiting poor misoprostol stability. The same lot after milling was found to have low residual acidity as shown in Table 2 and provided a dispersion having acceptable misoprostol stability.

### Example 3

A dispersion of 1 part misoprostol in 99 parts HPMC was prepared using a single lot of HPMC that was unmilled, milled by the supplier, or milled in the present applicants' laboratory. The HPMC lot used in this study was one known to result in poor misoprostol stability. A-form contents of the misoprostol dispersion following storage, together with loss-on-drying (LOD) data for the HPMC and pH of the sub-53 μm fraction of the HPMC as measured according to the procedure of Test I, are shown in Table 3.

TABLE 3

| | % A-form | HPMC | pH | weight % |
|---|---|---|---|---|
| Lot | content | LOD (%) | (sub-53 μm) | (sub-53 μm) |
| E (unmilled) | 5.7 | 3.08 | 3.2 | 48 |
| E (milled by | 0.17 | 2.84 | 6.6 | 65 |
| supplier) | | | | |
| E (laboratory | 0.08 | 1.56 | 7.7 | 78 |
| milled) | | | | |

Once again, milling of the HPMC caused a major increase in pH of the sub-53 μm fraction, and a great improvement in misoprostol stability as measured by low A-form content. Milling, probably as a result of generation of heat, also reduced the loss on drying of the HPMC. It is possible that this was an additional contributory factor in leading to improved misoprostol stability.

### Example 4

A second milling study was conducted with another HPMC lot known to result in poor misoprostol stability. A-form content data are shown in Table 4.

TABLE 4

| Lot | % A-form content |
|---|---|
| F (unmilled) | 1.31 |
| F (laboratory milled) | 0.06 |

### Example 5

Unmilled HPMC Lot E (as used in Example 3) was used to prepare a 1:99 misoprostol dispersion, the HPMC being either untreated or subjected to vacuum drying for 6 hours at 60° C. (two runs) or for 31 hours at 80° C. (one run). A-form content data are shown in Table 5.

TABLE 5

| Lot | % A-form content |
|---|---|
| E (not dried) | 5.24 |
| E (6 h, 60° C., first run) | 2.56 |
| E (6 h, 60° C., second run) | 1.47 |
| E (31 h, 80° C.) | 0.72 |

Vacuum drying of the unmilled HPMC resulted in improved misoprostol stability, but not to a degree sufficient to reduce A-form content to an acceptably low level.

### Example 6

Unmilled HPMC Lot E (as used in Example 3) was sieved to produce a sub-53 μm fraction and a “53-75 μm fraction”. Particle size analysis showed that a significant amount of sub-53 μm material remained in the “53-75 μm fraction”. Data for LOD, particle size analysis and pH are shown in Table 6, together with data for A-form content of a 1:99 misoprostol dispersion prepared with each HPMC fraction.

TABLE 6

| | | Particle size analysis | | | | | | A-form |
|---|---|---|---|---|---|---|---|---|
| | LOD | (%) | | | pH | | | content |
| Fraction | (%) | <53 μm | 53-75 μm | >75 μm | <53 μm | 53-75 μm | composite | (%) |
| sub-53 μm | 3.34 | 98.7 | 0.6 | 0.7 | 3.2 | n.d. | 3.2 | 8.6 |
| 53-75 μm | 3.75 | 36.1 | 63.3 | 0.6 | 3.4 | 6.4 | 5.7 | 0.81 |

n.d. = not determined

The data demonstrate that the major factor contributing to formation of A-form misoprostol resides in the<53 μm particle size component of the HPMC.

### Example 7

The sub-53 μm fraction of HPMC Lot E (as tested in Example 6) was blended with sodium bicarbonate in an amount of 2.5% or 5% by weight of the HPMC. A-form content of a 1:99 misoprostol dispersion was determined. Data are shown in Table 7.

TABLE 7

| Lot | % A-form content |
|---|---|
| E (sub-53 μm, no NaHCO₃) | 8.6 |
| E (sub-53 μm, 2.5% NaHCO₃) | 0.32 |
| E (sub-53 μm, 5% NaHCO₃) | 0.09 |

As shown in Table 7, addition of base to the HPMC provided a major improvement in misoprostol stability.

1. A discrete solid orally deliverable pharmaceutical dosage form comprising a plurality of zones, wherein (a) at least one zone comprises an NSAID, the NSAID being present in a therapeutically effective total amount in the dosage form; (b) at least one zone, other than a zone comprising the NSAID, comprises HPMC having dispersed therein a prostaglandin type compound in a form of a substantially water-free solid dispersion, the prostaglandin type compound being present in the dosage form in a total amount effective to mitigate a gastric ulcerogenic effect of the NSAID; (c) said plurality of zones are spatially arranged such that, if there is only one NSAID-containing zone and one prostaglandin-containing zone, said NSAID-containing and prostaglandin-containing zones are arranged other than as a core and mantle respectively having an enteric coating layer therebetween; and (d) said HPMC comprises a fraction having particle size smaller than about 53 μm, said fraction exhibiting, upon dissolution in CO₂-free purified water to form a 1% weight/volume solution, a pH not lower than about 4.

2. The dosage form of claim 1 having one NSAID-containing zone and one prostaglandin-containing zone.

3. The dosage form of claim 2 that is a bilayer tablet wherein said NSAID-containing zone and said prostaglandin-containing zone are disposed as layers.

4. The dosage form of claim 2 that is a dual-compartment capsule wherein said NSAID-containing zone and said prostaglandin-containing zone are disposed as compartments.

5. The dosage form of claim 2 wherein said NSAID-containing zone and said prostaglandin-containing zone are disposed as pre-compressed or pre-molded tablets embedded within a single larger dosage form.

6. The dosage form of claim 2 that is a tablet having a core comprising said prostaglandin type compound surrounded by a mantle comprising said NSAID.

7. The dosage form of claim 1 having more than two of said zones.

8. The dosage form of claim 7 that is a multilayer tablet wherein said at least one NSAID-containing zone and said at least one prostaglandin-containing zone are disposed as layers.

9. The dosage form of claim 8 comprising two outer prostaglandin-containing layers having sandwiched between them a middle NSAID-containing layer.

10. The dosage form of claim 7 wherein said zones are disposed as a plurality of separately NSAID-containing and prostaglandin-containing particles compressed or molded into a single tablet.

11. The dosage form of claim 7 that is a multi-compartment capsule wherein said at least one NSAID-containing zone and said at least one prostaglandin-containing zone are disposed as compartments.

12. The dosage form of claim 7 that is a capsule containing a plurality of separately NSAID-containing and prostaglandin-containing beads.

13. The dosage form of claim 7 that is a capsule containing a plurality of beads, at least a fraction of said beads individually comprising a core comprising said NSAID surrounded by a mantle comprising said prostaglandin type compound.

14. The dosage form of claim 1, further comprising a barrier layer having the effect of substantially preventing contact of the NSAID with the prostaglandin type compound.

15. The dosage form of claim 1 wherein said fraction of the HPMC exhibits, upon dissolution in CO₂-free purified water to form a 1% weight/volume solution, a pH not lower than about 4.5.

16. The dosage form of claim 1 wherein said fraction of the BPMC exhibits, upon dissolution in CO₂-free purified water to form a 1% weight/volume solution, a pH not lower than about 5.

17. The dosage form of claim 1 wherein said fraction of the HPMC exhibits, upon dissolution in CO₂-free purified water to form a 1% weight/volume solution, a pH not lower than about 6.

18. The dosage form of claim 1 wherein said prostaglandin type compound is selected from prostaglandins E₁ and E₂ and derivatives thereof.

19. The dosage form of claim 1 wherein said prostaglandin type compound is misoprostol.

20. The dosage form of claim 19 wherein said misoprostol is dispersed in said HPMC in a weight ratio of about 1:1000 to about 1:10.

21. The dosage form of claim 19 wherein said misoprostol is dispersed in said HPMC in a weight ratio of about 1:500 to about 1:20.

22. The dosage form of claim 19 wherein said misoprostol is dispersed in said HPMC in a weight ratio of about 1:200 to about 1:50.

23. The dosage form of claim 19 wherein said misoprostol is present in the dosage form in a total amount of about 50 to about 400 μg.

24. The dosage form of claim 19 wherein said misoprostol is present in the dosage form in a total amount of about 100 to about 300 μg.

25. The dosage form of claim 1 wherein said NSAID is selected from diclofenac and its pharmaceutically acceptable salts, and piroxicam.

26. The dosage form of claim 1 wherein said NSAID is diclofenac sodium.

27. The dosage form of claim 26 wherein said diclofenac sodium is present in the dosage form in a total amount of about 20 to about 200 mg.

28. The dosage form of claim 26 wherein said diclofenac sodium is present in the dosage form in a total amount of about 40 to about 100 mg.

29. The dosage form of claim 1 wherein said NSAID is diclofenac sodium in a total amount of about 50 mg and said prostaglandin type compound is misoprostol in a total amount of about 200 μg.

30. The dosage form of claim 1 wherein said NSAID is diclofenac sodium in a total amount of about 75 mg and said prostaglandin type compound is misoprostol in a total amount of about 200 μg.

31. An assay method for selecting lots of HPMC suitable for use in preparing a solid dispersion of a prostaglandin type compound, the method comprising (a) fractionating a sample of a test lot of HPMC by particle size to provide a sub-53 μm fraction; (b) dissolving said fraction in CO₂-free purified water to provide a 1% weight/volume solution; (c) measuring pH of said solution; and (d) selecting the test lot for said use if the pH is 4.0 or higher.

32. A process for preparing a solid dispersion of a prostaglandin type compound in HPMC comprising a step of selecting a lot of HPMC by the assay method of claim 31.

33. A process for preparing a dosage form of claim 1 comprising a step of selecting a lot of HPIMC by an assay method that comprises (a) fractionating a sample of said lot of HPMC by particle size to provide a sub-53 μm fraction; (b) dissolving said fraction in CO₂-free purified water to provide a 1% weight/volume solution; (c) measuring pH of said solution; and (d) selecting the test lot for preparing said dosage form if the pH is 4.0 or higher.

34. A process for preparing a dosage form of claim 1 comprising a step of treating said HPMC such that, following such treatment, a fraction of said HPMC having particle size smaller than about 53 μm exhibits, upon dissolution in CO₂-free purified water to form a 1% weight/volume solution, a pH not lower than about 4.

35. The process of claim 34 wherein said treatment comprises milling the HPMC.

36. The process of claim 34 wherein said treatment comprises adding a pH modifying agent to the HPMC.

37. The process of claim 36 wherein the pH modifying agent is a base.

38. The process of claim 34 wherein said treatment comprises vacuum drying the HPMC.
